(19) United States
(12) Patent Application Publication
(10) Pub. No.: US 2014/0316391 A1
(43) Pub. Date: Oct. 23, 2014

(54) EYESIGHT CORRECTION APPARATUS AND METHOD FOR CONTROLLING SAME

(71) Applicant: Lutronic Corporation, Gyeonggi-do (KR)

(72) Inventor: Hee Chul Lee, Gyeonggi-do (KR)

(21) Appl. No.: 14/360,510

(22) PCT Filed: Nov. 26, 2012
(86) PCT No.: PCT/KR2012/010075

§ 371 Date: May 23, 2014

(30) Foreign Application Priority Data
Nov. 24, 2011 (KR) 10-2011-0123562

(51) Int. Cl. A61F 9/008

(52) U.S. Cl. CPC A61F 9/00821; A61F 2009/00851; A61F 2009/00872
USPC 606/5

(57) ABSTRACT

The eyesight correction apparatus according to the present invention comprises: a cutting unit for cutting out a portion of the cornea such that a stromal bed and a flap are arranged; an image processing unit for collecting, in images, the state between the flap which is located on the stromal bed and processing the images; a beam generating unit for generating a joining beam for interconnecting between the flap and a cut area of the cornea on the basis of the image signal processed by the image processing unit; a beam delivery unit for guiding the joining beam along the cut area between the flap and the cornea; and a control unit for controlling an operation of the beam delivery unit such that the joining beam can be radiated along the cut area between the flap and the cornea on the basis of the image signal.

### BACKGROUND OF THE INVENTION

1. Field of the Invention

The present invention relates to an vision correction apparatus and a method of controlling the same, and more particularly an vision correction apparatus that corrects eyesight, using a laser and a method of controlling the vision correction apparatus.

2. Related Art

Recently vision correction apparatuses that satisfy various types of operations for correcting eyesight have been developed. In addition to vision correction apparatuses that can satisfy the operation types, for example, LASIK (laser in situ keratomileusis) and LASEK (laser assisted sub-epithelial keratomileusis), vision correction apparatuses that can perform various operations such as wavefront LASIK, epi-LASIK, and i-LASIK, which are included in LASIK, have been developed.

The vision correction apparatus performing an operation in the type of LASIK in the vision correction apparatuses is characterized in that it creates a flap by cutting off a portion of a cornea and radiating an vision correction beam to the stromal bed that is the portion with the flap separated, thereby correcting eyesight. The separated flap is arranged to cover the stromal bed after vision correction.

As an vision correction apparatus of the related art, there is a “device for separation of corneal epithelium” disclosed in Korean Patent Application Publication No. 2006-0097709. The prior art document provides a device for the LASIK surgery, which includes a handpiece having a traverse motor and a vibrator motor for creating a flap separated from a portion of a cornea.

However, the device has a problem in that it uses a handpiece for cutting off a portion of a cornea to perform the LASIK surgery, but the flap separated by the handpiece is arranged simply cover the stromal bed, such that the flap may be separated, when impact is applied the cornea.

### SUMMARY OF THE INVENTION

The present invention has been made in an effort to provide an vision correction apparatus that improves the type of an vision correction operation so that a flap separated from a cornea can be permanently supported on the cornea, and a method of controlling the vision correction apparatus.

An vision correction apparatus according to the present invention includes: a cutting-off unit that cuts off a portion of a cornea to provide a stromal bed to be corrected and a flap partially separated from the cornea; an image processing unit that an image processing unit which collects and processes images about states between the cornea and the flap positioned over the stromal bed and covering the stromal bed after the stromal bed is corrected; a beam generating unit that generates a welding beam for welding the cut portion between the flap and the cornea on the basis of an image signal processed by the image processing unit; a beam delivery unit that guides the welding beam generated by the beam generating unit along the cut portion of the flap and the cornea; and a control unit that controls the operation of the beam delivery unit so that the welding beam is radiated along the cut portion between the flap and the cornea, on the basis of the image signal processed by the image processing unit.

The image processing unit may include: an image collecting part that collects images about the cutting status of the cornea when the cornea is cut off, and collects image about the welding status when the cornea and the flap are welded; and an image processing part that processes images from the image collecting part and transmit them to the control unit.

Preferably, the image processing unit may include an optical coherent tomography.

The cutting-off unit may include a microkeratome.

On the other hand, it is preferable that the cutting-off unit radiates a femtosecond laser to the cornea.

The welding beam generated by the beam generating unit may include a femtosecond laser.

The vision correction apparatus may further include an objective lens that is disposed between the cornea and the beam generating unit and concentrates the welding beam generated by the beam generating unit.

Further, the vision correction apparatus may further include a regulating unit that regulates the distance between the cornea and the objective lens.

A method of controlling an vision correction apparatus according to the present invention includes: (a) cutting off a portion of a cornea to create a flap that is a portion separated from the cornea; (b)covering a stromal bed with the flap and radiating a welding beam to the cut portion of the cornea and the flap, after the stromal bed with the flap separated is corrected; and (c) controlling the radiation position of the welding beam so that the welding beam is radiated along the cut portion of the cornea and the flap, when the cornea and the flap are welded.

The step (c) may include collecting and processing images about the welding status of the cornea and the flap and the radiation position of the welding beam, when the cornea and the flap are welded.

Preferably, the step (a) may use any one of a microkeratome and a femtosecond laser.

The vision correction apparatus may include an objective lens that concentrates the welding beam radiated to the cornea.

The step (b) may include regulating the distance between the cornea and the objective lens.

The welding beam may include a femtosecond laser.

The details of other embodiments are included in the following detailed description and the accompanying drawings.

According to the vision correction apparatus and a method of controlling the vision correction apparatus of the present invention, it is possible to prevent the flam from being separated by an external impact by welding the cut portion between the cornea and the flap separated from the cornea with the welding beam, and thus it is possible to the patient's satisfaction at the operation.

### BRIEF DESCRIPTION OF THE DRAWINGS

FIG. 1 is a control block diagram of a schematic configuration of an vision correction apparatus according to an embodiment of the present invention.

FIGS. 2A to 2C are perspective views illustrating the operation of an vision correction apparatus according to a first embodiment of the present invention.

FIGS. 3A to 3C are perspective views illustrating the operation of an vision correction apparatus according to a second embodiment of the present invention.

FIG. 4 is a flowchart illustrating control of the vision correction apparatuses according to the first and second embodiments of the present invention.

### DESCRIPTION OF EXEMPLARY EMBODIMENTS

Hereinafter, vision correction apparatuses according to embodiments of the present invention and a method of controlling the vision correction apparatuses are described in detail with reference to the accompanying drawings.

Before the description, different eyesight apparatuses of the present invention will be described in the first and second embodiments, but it should be understood that the components in the configuration of the vision correction apparatuses according to the first and second embodiments are given the same names and reference numerals.

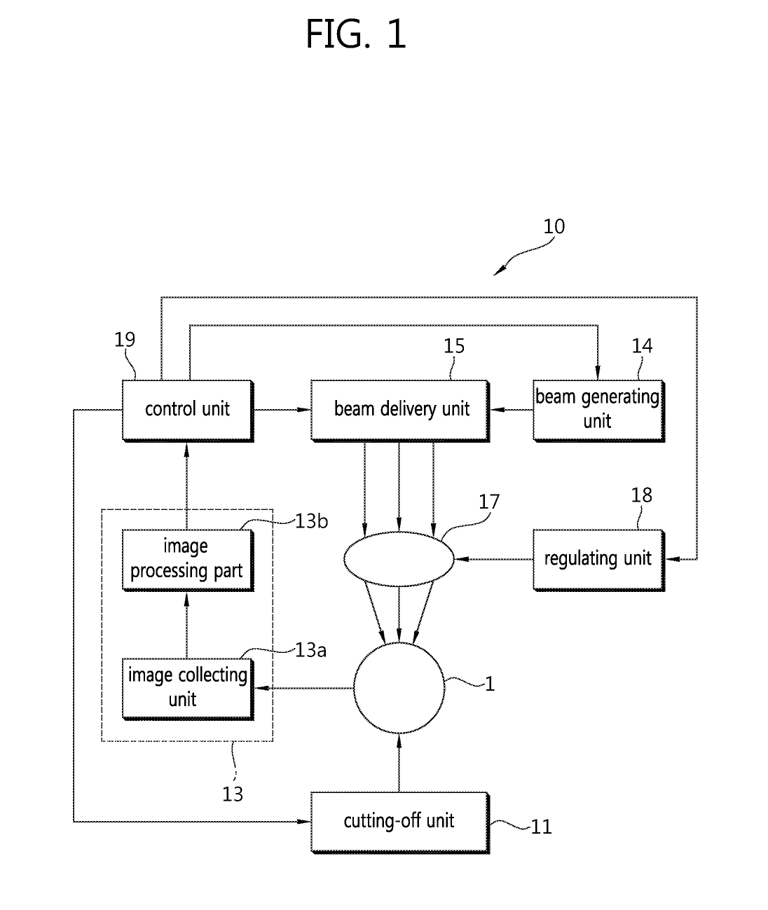

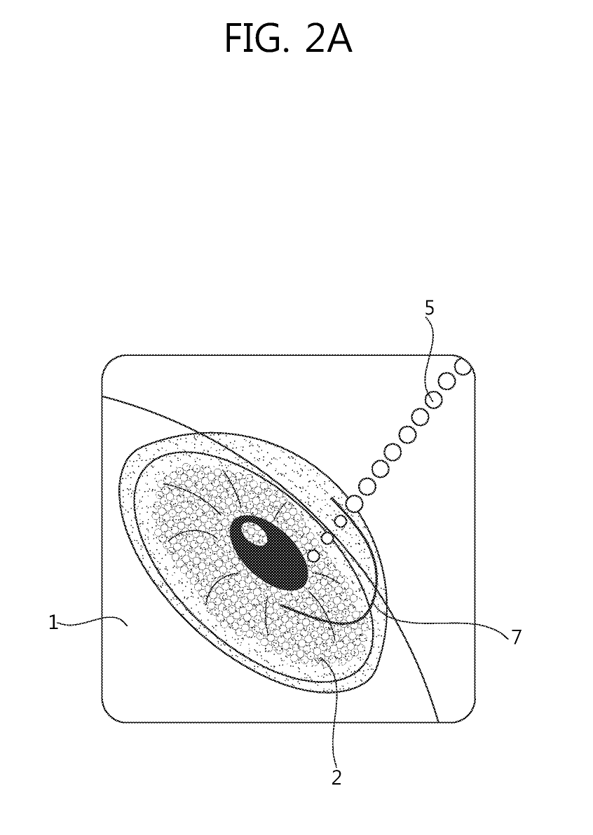

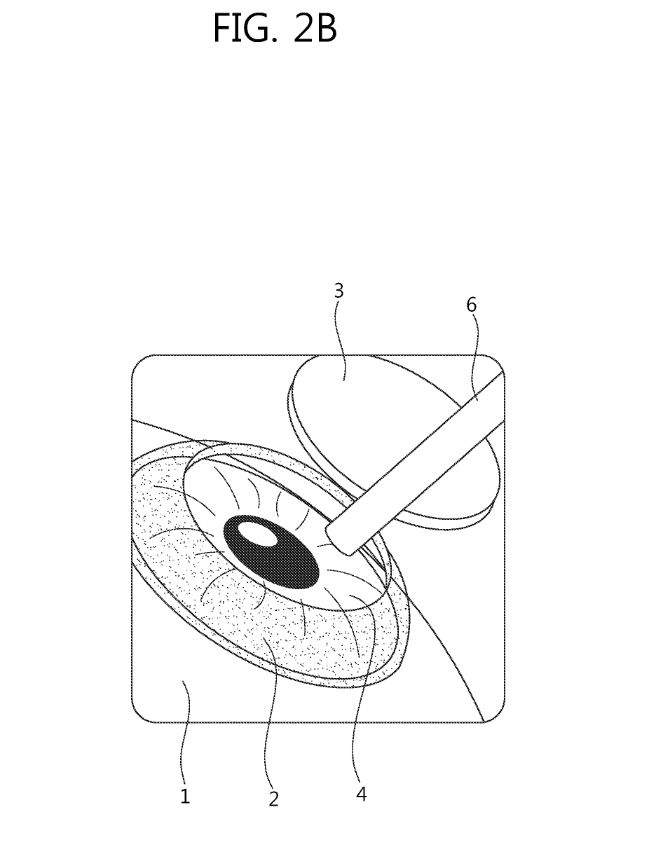

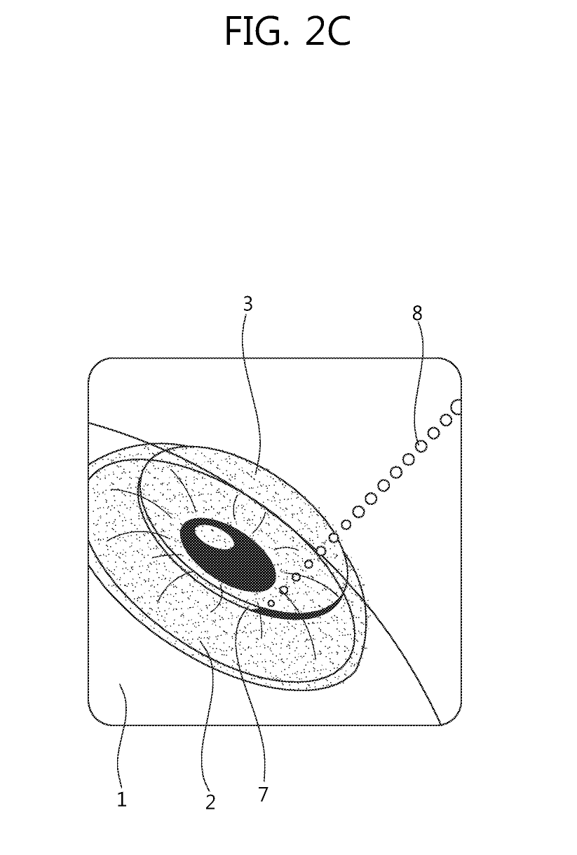

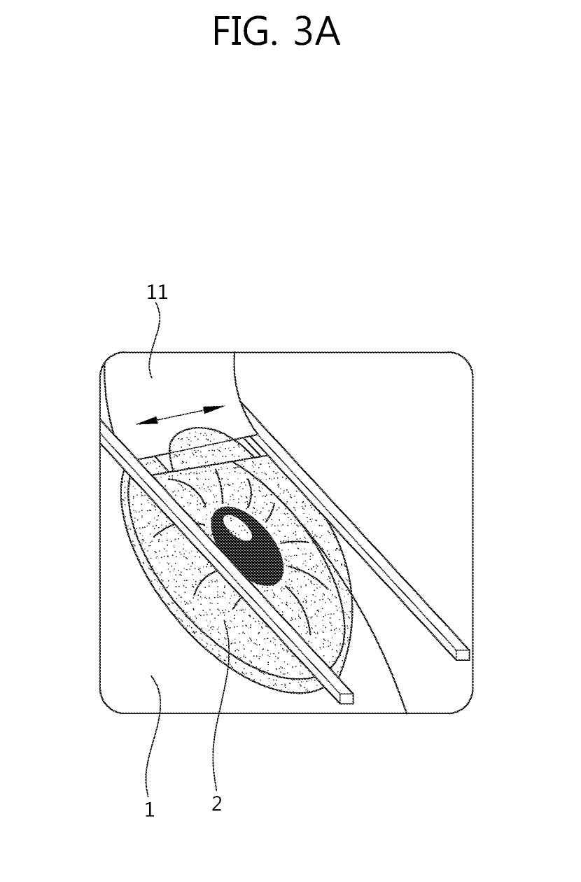

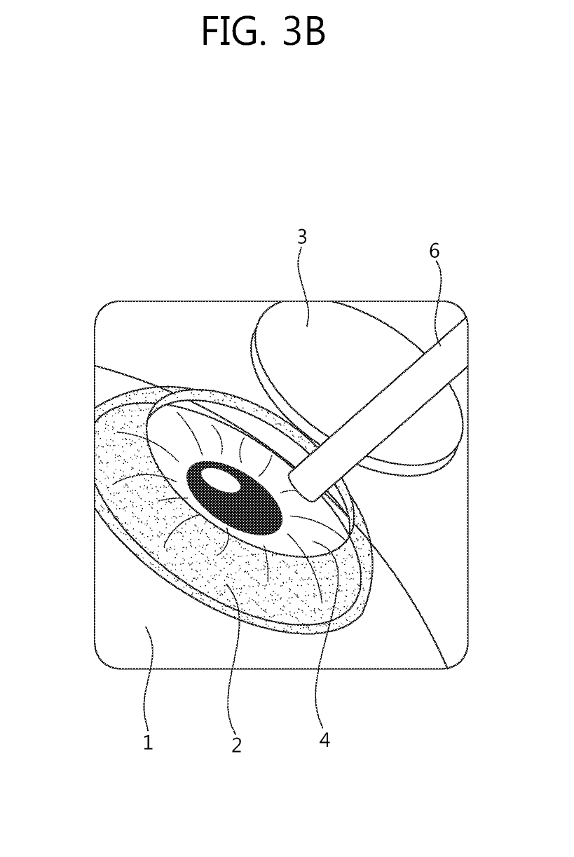

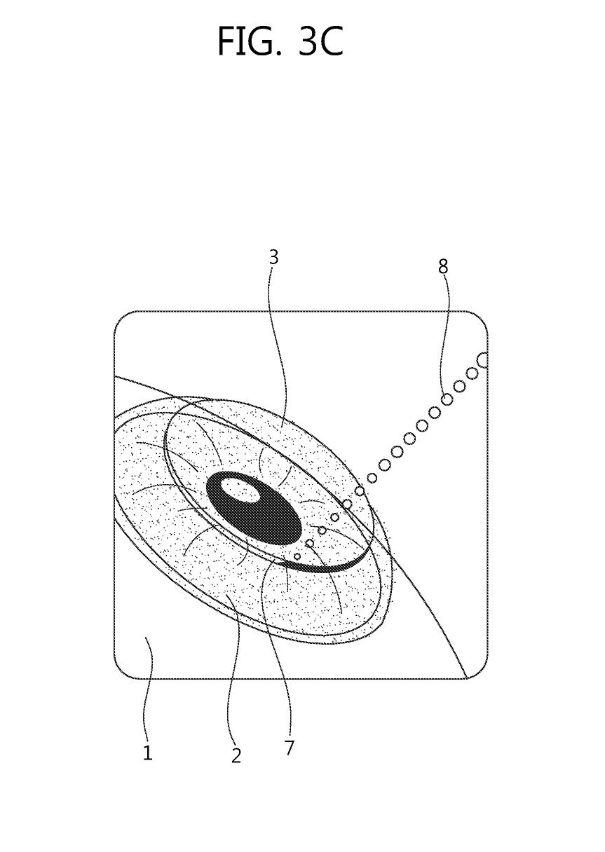

FIG. 1 is a control block diagram of a schematic configuration of an vision correction apparatus according to an embodiment of the present invention, FIGS. 2A to 2C are perspective views illustrating the operation of an vision correction apparatus according to a first embodiment of the present invention, and FIGS. 3A to 3C are perspective views illustrating the operation of an vision correction apparatus according to a second embodiment of the present invention.

As shown in FIGS. 1 to 3C, an vision correction apparatus 10 according to an embodiment of the present invention includes a body (not shown), a cutting-off unit 11, an image processing unit 13, a beam generating unit 14, a beam delivery unit 15, an objective lens 17, a regulating unit 18, and a control unit 19. The vision correction apparatus 10 according to an embodiment of the present invention is used for a LASIK (laser in-situ keratomileusis) surgery.

The body forms the external shape of the vision correction apparatus 10 and receives or is equipped with the cutting-off unit 11, the image processing unit 13, the beam generating unit 14, the beam delivery unit 15, the objective lens 17, the regulating unit 18, and the control unit 19.

The cutting-off unit 11 cuts off a portion of a cornea 2 so that an vision correction beam 6 can be radiated to the stromal bed 4, that is, the stroma of the cornea. A flap 3, which is the portion of the cornea 2 cut off by the cutting-off unit 11 is cut off from the cornea 2. The flap 3 cut off by the cutting-off unit 11 is not completely separated from the cornea 2, but connected to a portion of the cornea 1 through a small area. The flap 3 formed by the cutting-off unit 11 has an epithelium and a bowman's membrane.

The cutting-off unit 11 according to the first embodiment of the present invention cuts off the cornea 2, using a femtosecond laser that is a cutting-off beam 5. That is, as shown in FIG. 2A, the cutting-off unit 11 cuts off the cornea 2 to create the flap 3 that is separated from the cornea 2 by radiating a femtosecond laser in a closed loop shape to an area on the cornea 2. The cutting-off unit 11 according to the second embodiment of the present invention is a part independent from the beam generating unit 14 in FIG. 1, but it may be integrated with the beam generating unit 14.

The cutting-off unit 11 according to the second embodiment of the present invention is microkeratome using a blade to cut off the cornea 2. That is, as shown in FIG. 3A, the cutting-off unit 11 that is a microkeratome is moved over the cornea 2 and cuts off a portion of the cornea 2 to create the flap 3 to be separated from the cornea 2.

The vision correction apparatuses according to the first and second embodiments of the present invention, as shown in FIGS. 2B and 3B, radiate the vision correction beam 6 to the stromal bed 4 formed by the cutting-off unit 11. The configuration that radiates the vision correction beam 6 may be integrated with the beam generating unit 14 that radiates a welding beam 8 or may be provided independently from the beam generating unit 14.

Next, the image processing unit 13 collects and processes image so that the operation state of an eyeball 1 can be monitored, when the cutting-off unit 11 cuts the cornea 2 and the beam generating unit 4 and the beam delivery unit 15 are operated to weld the cut portion 7 of the flap 3 to the cornea 2. For example, the image processing unit 13 collects and processes images about the cutting depth and the cutting range of the cornea 2, when the cornea 2 is cut off, and it collects and processes an image about the welding status of the cut portion 7, when the flap 3 is welded to the cornea 2.

The image processing unit 13 of the present invention includes an image collecting part 13a that collects images of the eyeball and an image processing part 13b that processes the images collected by the image collecting unit 13a. The processing signals by the image processing unit 13b are transmitted to the control unit 19 so that the beam generating unit 14 and the beam delivery unit 15 can be controlled.

The image processing unit 13 of the present invention includes an OCT (Optical Coherence Tomography). The OCT that is the image processing unit 13 can measure distances from the wavelengths of light reflecting from different structures of the eyeball 1, using short coherent light, and collect and processes high-resolution transverse images. Thee image processing unit 13 that is an OCT can induce a more precise operation by collecting and processing images in real time with cutting-off and welding of the cornea 2 and transmitting them to the control unit 19.

The beam generating unit 14 generates a welding beam 8 for welding the cut portion 7 between the cornea 2 and the flap 3 on the basis of the image signals processed by the image processing unit 13, when the cornea 2 and the flap 3 are welded. The welding unit 8 generated by the beam generating unit 14 is a femtosecond laser.

The beam delivery unit 15 guides the beam generating unit 14 along the cut portion of the cornea 2 and the flap 3, as shown in FIGS. 2C and 3C. The beam delivery unit 15 includes a scanner that can adjust the radiation position of the welding beam 8 generated by the beam generating unit 14. The beam delivery unit 15 is controlled by the control unit 19 such that the welding beam 8 is radiated to appropriate positions along the cut portion 7 of the cornea 2 and the flap 3 or in accordance with the cutting depth.

The wavelength of the welding beam 8 guided by the beam delivery unit 15 has a band where a large amount of welding beam is absorbed in the cornea 2. When the wavelength band of the welding beam 8 is too high, too much beam is absorbed to the surface of the cornea 2, such that there is a need of selecting a wavelength band having an appropriate absorption ratio in order to achieve the required result. Further, the pulse width of the welding beam 8 should not be larger than TRT (Thermal Relaxation Time) of the corneal tissue, so it should be smaller than the TRT in consideration of the TRT. The size of the spot formed by the welding beam should be determined over tens of micrometers, with the cut surface between the cornea 2 and the flap 3 seen by the OCT. The welding beam 8 is radiated to the tissue of the cut surface between the cornea 2 and the flap 3 under the conditions described above and welds the cornea 2 and the flap 3 together by increasing the temperature of the tissue to a level where the tissue is degenerated.

The objective lens 17 is disposed between the cornea 2 and the beam generating unit 14 and concentrates the welding beam 8 generated by the beam generating unit 14. The objective lens 17 is disposed substantially at one side of the body which is moved close to the cornea 2. The objective lens 17 can increase the energy provided to the cut portion 7 between the cornea 2 and the flap 3 by concentrating the welding beam 8 generated by the beam generating unit 14.

The regulating unit 18 is provided to regulate the distance between the objective lens 17 and the cornea 2. The regulating unit 18 may be a lens barrel such as a camera or other parts such as a motor known in the art. The regulating unit 18 regulates the distance between the cornea 2 and the objective lens 17 so that the cornea 2 and the flap 3 can be easily welded.

Finally, the control unit 19 controls the operation of the beam delivery unit 15 to adjust the radiation position of the welding beam 8 radiated to the cut portion 7 between the cornea 2 and the flap 3 on the basis of signals from the image processing unit 13. That is, the control unit 19 controls the operation of the beam delivery unit 15 that can change the radiation path of the welding beam 8 so that the welding beam 8 is radiated along the cut portion 7 between the cornea 2 and the flap 3 by analyzing image signals transmitted in real time from the image processing unit 13. The control unit 19 can control the cutting-off unit 11 that cuts off the cornea 2, on the basis of image signals from the image processing unit 13, in an operation of cutting off the cornea 2.

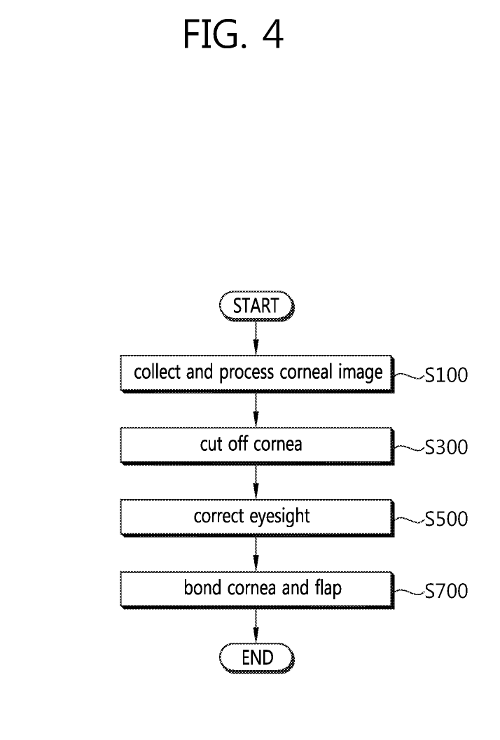

FIG. 4 is a flowchart illustrating control of the vision correction apparatuses 10 according to the first and second embodiments of the present invention.

A method of controlling the vision correction apparatuses 10 according to the first and second embodiments of the present invention which have the configurations described above are described hereafter with reference to FIG. 5.

First, images of the eyeball 1 are collected and processed to cut off the cornea 2 (S100). The cutting-off unit 11 cuts off the cornea 2 to form the flap 3 that is a portion separated from the cornea 2 (S300). The cutting-off unit 11 may use a femtosecond laser, as in the first embodiment, or may use a microkeratome including a blade, as in the second embodiment.

When the cornea 2 is cut off and the flap 3 is obtained, the vision correction beam 6 is radiated to the stromal bed 4 that has been covered by the flap 3 (S500). The vision correction beam 8 radiated to the stromal bed 4 of the eyeball 1 can make the stromal bed 4 flat and can change the curvature of the stromal bed 4.

After the vision correction beam 6 is radiated and the vision correction is finished, the stromal bed 4 is covered by the separated flap 3. The cornea 2 and the flap 3 are welded by radiating the welding beam 8 to the cut portion 7 between the cornea 2 and the flap 3 on the basis of an image signal from the image processing unit (S700). The welding of the cut portion 7 between the cornea 2 and the flap 3 can be achieved by controlling the beam delivery unit.

Accordingly, it is possible to prevent the flam from being separated by an external impact by welding the cut portion between the cornea and the flap separated from the cornea with the welding beam, and thus it is possible to the patient's satisfaction at the operation.

Although embodiments of the present invention were described above with reference to the accompanying drawings, those skilled in the art would understand that the present invention may be implemented in various ways without changing the necessary features or the spirit of the prevent invention. Therefore, the embodiments described above are only examples and should not be construed as being limitative in all respects. The scope of the present invention is defined by not the specification, but the following claims, and all of changes and modifications obtained from the meaning and range of claims and equivalent concepts should be construed as being included in the scope of the present invention.

1. An vision correction apparatus comprising:
a cutting-off unit which cuts off a portion of a cornea to provide a stromal bed to be corrected and a flap which is a portion separated from the cornea;
an image processing unit which collects and processes images about states between the cornea and the flap positioned over the stromal bed and covering the stromal bed after the stromal bed is corrected;
a beam generating unit which generates a welding beam for welding the cut portion between the flap and the cornea on the basis of an image signal processed by the image processing unit;
a beam delivery unit which guides the welding beam generated by the beam generating unit along the cut portion of the flap and the cornea; and
a control unit which controls the operation of the beam delivery unit so that the welding beam is radiated along the cut portion between the flap and the cornea, on the basis of the image signal processed by the image processing unit.

2. The vision correction apparatus of claim 1, wherein the image processing unit comprises:
an image collecting part which collects images about the cutting status of the cornea when the cornea is cut off, and collects image about the welding status when the cornea and the flap are welded; and
an image processing part which processes images from the image collecting part and transmits them to the control unit.

3. The vision correction apparatus of claim 2, wherein the image processing unit includes an optical coherent tomography (OCT).

4. The vision correction apparatus of claim 1, wherein the cutting-off unit includes a microkeratome.

5. The vision correction apparatus of claim 1, wherein the cutting-off unit radiates a femtosecond laser to the cornea.

6. The vision correction apparatus of claim 1, wherein the welding beam generated by the beam generating unit includes a femtosecond laser.

7. The vision correction apparatus of claim 6, further comprising an objective lens which is disposed between the cornea and the beam generating unit and concentrates the welding beam generated by the beam generating unit.

8. The vision correction apparatus of claim 6, further comprising a regulating unit which regulates the distance between the cornea and the objective lens.

9. A method of controlling an vision correction apparatus, the method comprising:
(a) cutting off a portion of a cornea to create a flap which is a portion separated from the cornea;
(b) covering a stromal bed with the flap and radiating a welding beam to the cut portion of the cornea and the flap, after the stromal bed with the flap separated is corrected; and
(c) controlling the radiation position of the welding beam so that the welding beam is radiated along the cut portion of the cornea and the flap, when the cornea and the flap are welded.

10. The method of claim 9, wherein the step (c) includes collecting and processing images about the welding status of the cornea and the flap and the radiation position of the welding beam, when the cornea and the flap are welded.

11. The method of claim 9, wherein the step (a) uses any one of a microkeratome and a femtosecond laser.

12. The method of claim 9, wherein the vision correction apparatus includes an objective lens which concentrates the welding beam radiated to the cornea.

13. The method of claim 12, wherein the step (b) includes regulating the distance between the cornea and the objective lens.

14. The method of claim 9, wherein the welding beam includes a femtosecond laser.
